(19) United States
(12) Patent Application Publication
(10) Pub. No.: US 2012/0196936 A1
(43) Pub. Date: Aug. 2, 2012

(54) Composition Comprising S-Allyl-L-Cysteine as Active Ingredient for Preventing or Treating Gastrointestinal Disorders

(75) Inventors: Soon Bae Kim, Gyeonggi-Do (KR); Gwang Soon Kim, Gyeonggi-Do (KR); Wan Bae Kim, Seoul (KR); Wie Jong Kwak, Seoul (KR); Sung Hye Bang, Seoul (KR)

(73) Assignee: PHARMAKING CO., LTD., Chungcheongbuk-Do (KR)

(21) Appl. No.: 13/497,421

(22) PCT Filed: Sep. 20, 2010
(86) PCT No.: PCT/KR2010/006506

§ 371 Date: Apr. 16, 2012

(30) Foreign Application Priority Data
Sep. 23, 2009 (KR) 10-2009-0090232

(51) Int. Cl. A61K 31/195; A61P 35/00; A61P 1/04; C07C 323/58; A61P 1/00

(52) U.S. Cl. 514/562; 562/557

(57) ABSTRACT

A composition including S-allyl-L-cysteine as an active ingredient and having an anti-Helicobacter pylori activity or a gastric mucosa protective effect, a composition for preventing, relieving, or treating gastrointestinal disorders, and a method of using the compositions.

### CROSS-REFERENCE TO RELATED PATENT APPLICATION

This application claims the benefit of Korean Patent Application No. 10-2009-0090232, filed on Sep. 23, 2009, in the Korean Intellectual Property Office, the disclosure of which is incorporated herein in its entirety by reference.

### TECHNICAL FIELD

1. Field of the Invention

The present invention relates to a composition having an anti-Helicobacter pylori activity and a gastric mucosa protective effect, a composition for preventing or treating gastrointestinal disorders, and a method of using the compositions.

2. Background Art

Gastrointestinal disorders are caused by a variety of factors and are known to be caused by an imbalance between aggressive factors, such as Helicobacter pylori, gastric acid, pepsin, overwork, stress, and alcohol,and defensive factors, such as mucus secretion, tissue-regenerative capability, and anticoagulant activity. Gastritis caused by overwork, stress, Helicobacter pylori infection, or the like is a common symptom but can develop, if not treated, into chronic gastritis, gastric ulcer, and, rarely, gastric cancer. A gastric mucosal lesion caused by alcohol can be healed within several days by the removal of the stimulus factor but may develop into gastrointestinal bleeding, a gastric perforation, or the like (Taeyoung Oh, et al., J. Applied Pharmacology, Vol. 5, pp 202-210, 1997). Various drugs such as cimetidine, ranitidin, famotidine, omeprazole, or bismuth have been used for treating gastrointestinal disorders. However, the relapse rate after discontinuation of these drugs is very high, and thus there is a need to develop a novel drug.

Helicobacter pylori is a gram-negative bacterium that colonizes human gastric mucosa or mucus. It is recognized that Helicobacter pylori infection is a significant contributory factor in the development of most gastrointestinal tract-associated disorders such as acutechronic gastritis, atrophic gastritis, gastric ulcer, gastric cancer, and duodenal ulcers (Crowe, Curr Opin Gastrenterol., 21(1), pp 32-38, 2005). Furthermore, it has been reported that Helicobacter pylori infection is a risk factor for hepatic encephalopathy, arteriosclerosis, and hepatobiliary system-associated diseases in addition to digestive system-associated diseases (Karahalil, et al., Curr Drug Saf, 2, pp 43-46, 2007; Scragg, et al., J. Epidemiol Community Health, 50(5), pp 578-579, 1996). According to the US Centers for Disease Control and Prevention (CDC), Helicobacter pylori infection may cause chronic fatigue, urticaria, migraine, short stature, infertility, food allergy, etc., which are all symptoms of ‘strange Helicobacter syndrome’. In general, antibiotics are used to kill Helicobacter pylori. However, Helicobacter pylori reinfection is common after eradication thereof and a high-dose treatment is required for a long period of time to completely remove the Helicobacter pylori. Accordingly,the long-term use of high dose antibiotics may lead to side effects and increase in antibiotic-resistant bacteria.

Recently, diverse research is being conducted into materials that inhibit the growth of bacteria in food as a safe method to treat Helicobacter pylori infection-associated diseases. Lactobacillus-fermented milk using probiotics, egg-yolk-derived immunoglobulin (IgY) including a Helicobacter pylori-neutralizing antibody, and catechin contained in wine and green tea are considered as effective substances against infection by Helicobacter pylori (McMahon, et al., Aliment Pharmacol Ther 23(8), pp 1215-1223, 2006; Sachdeva, et al., Eur J Gastroenterol Hepatol, 21(1), pp 45-53, 2009; Shin, et al., J Med Microbiol., 53(Pt 1), pp 31-34, 2004).

Meanwhile, garlic (Allium Sativum.L) that belongs to the Allium genus has antimicrobial, antifungal, anti-oxidant, and anti-cancer properties (Ankri, et al., Microbes Infect. 1(2), pp 125-129, 1999), prevents thrombosis, inflammation, and oxidative stress of cells (Sener, et al., Mol Nutr Food Res., 51(11), pp 1345-1352, 2007), and thus has drawn attention. Garlic contains various components including steroidal saponin such as eruboside-B having antifungal and anti-cancer properties (Matsuura H, et al., Chem Pharm Bull (Tokyo), 36: 3659-3663, 1988), glycoside fractions having a cholesterol-lowering effect (Slowing, et al., J Nutr., 131, pp 994S-9S, 2001), nonsulfur compounds such as β-chlorogenine having a platelet aggregation inhibiting effect (Rahman K, et al., J. Nutr. 2006), and various organosulfur compounds. Examples of the organosulfur compounds contained in plants belonging to the Allium genus are fat-soluble organosulfur compounds such as S-allyl-L-cysteine sulfoxides (alliin), Dially-disulfide (DADS), and Diallyl sulfide (DAS) and water-soluble organosulfur compounds such as S-allyl-L-cysteine (SAC) and S-allylmercaptocysteine (SAMC).

It has been reported that SAC, which is an active ingredient of mature garlic, has an anti-oxidant activity that inhibits arteriosclerosis and anticancer activity in some cancer cell lines (Proceedings of the American Association for Cancer Research, 30, p 181, 1989). However, it has not been reported that SAC has therapeutic effects on gastrointestinal diseases and an anti-Helicobacter pylori activity.

### DISCLOSURE OF INVENTION

### Technical Problem

In general, antibiotics are used to kill Helicobacter pylori. However, Helicobacter pylori reinfection is common after eradication thereof and high-dose treatment is required for a long period of time to completely remove the Helicobacter pylori. Accordingly, side effects may occur and antibiotic-resistant bacteria may increase due to the use of antibiotics. Therefore, there is a need for an alternative drug other than antibiotics. The present invention provides a drug having an anti-Helicobacter pylori activity and a gastric mucosa protective effect. The present invention also provides a safe composition for preventing, relieving, or treating gastrointestinal disorders.

### Solution to Problem

According to an aspect of the present invention, there is provided a composition comprising S-allyl-L-cysteine (SAC), which is a water-soluble organosulfur compound contained in a plant belonging to the Allium genus, as an active ingredient and having an anti-Helicobacter pylori activity and a mucosa protective effect. According to another aspect of the present invention, there is provided a composition including SAC as an active ingredient for preventing, relieving, or treating gastrointestinal disorders.

### ADVANTAEOUS EFFECTS OF INVENTION

The composition including SAC as an active ingredient according to the present invention inhibits Helicobacter pylori infection and protects against gastric lesions caused by Helicobacter pylori. Accordingly, the composition may be used as an anti-Helicobacter pylori drug.

The composition including SAC as an active ingredient according to the present invention prevents and treats gastric mucosal lesions caused by hydrochloric acid-ethanol, aspirin, or indomethacin, and thus may be efficiently used to prevent, relieve, or treat gastrointestinal disorders. The composition including SAC as an active ingredient according to the present invention may be used as a pharmaceutical composition or food composition.

### BRIEF DESCRIPTION OF DRAWINGS

The above and other features and advantages of the present invention will become more apparent by describing in detail exemplary embodiments thereof with reference to the attached drawings in which:

FIG. 1 shows average weights of animals of control and experimental groups for the entire test period, wherein all values are shown as averages;

FIGS. 2A and 2B show an effect of S-allyl-L-cysteine (SAC) on average serum IgG antibody production of animals of control and experimental groups measured after Helicobacter pylori infection, wherein all values are shown as averages and standard errors, **: P<0.01, and *: P<0.05;

FIG. 3 shows an effect of SAC on average serum TNF-α of animals of control and experimental groups measured for an entire 10-week test period, wherein all values are shown as averages and standard errors;

FIG. 4 shows tissue pathological changes of animals of control and experimental groups;

FIG. 5 shows tissue pathological changes and the number of eosinophils of animals of control and experimental groups, wherein all values are shown as averages and standard errors, **: P<0.01, and *: P<0.05;

FIG. 6 shows tissue pathological changes (mitotic figures) of animals of control and experimental groups, wherein all values are shown as averages and standard errors;

FIG. 7 shows serum glutamic oxaloacetic transaminase (GOT) levels of animals of control and experimental groups, wherein all values are shown as averages and standard errors;

FIG. 8 shows serum glutamate pyruvate transaminase (GPT) levels of animals of control and experimental groups, wherein all values are shown as averages and standard errors;

FIG. 9 shows serum copper and zinc containing-superoxide dismutase (Cu/Zn-SOD) levels of animals of control and experimental groups, wherein all values are shown as averages and standard errors;

FIG. 10 shows an effect of SAC on lengths of gastric lesions of rats induced by hydrochloric acid-ethanol, wherein ** indicates a significant difference from a vehicle control group G1 by p<0.01, G1: vehicle control group (distilled water), G2: 100 mg/kg of SAC, G3: 200 mg/kg of SAC, G4: 400 mg/kg of SAC, and G5: positive control group (55.6 mg/kg of Stillen® as an active ingredient);

FIG. 11 shows gastric lesion inhibiting rates in rats in hydrochloric acid-ethanol-induced gastric lesion model, wherein ** indicates a significant difference from a vehicle control group G1 by p<0.01, G1: vehicle control group (distilled water), G2: 100 mg/kg of SAC, G3: 200 mg/kg of SAC, G4: 400 mg/kg of SAC, and G5: positive control group (55.6 mg/kg of Stillen® as an active ingredient);

FIG. 12 shows photographs of stomachs of rats in hydrochloric acid-ethanol-induced gastric lesion model, wherein G1: vehicle control group (distilled water), G2: 100 mg/kg of SAC, G3: 200 mg/kg of SAC, G4: 400 mg/kg of SAC, and G5: positive control group (55.6 mg/kg of Stillen® as an active ingredient);

FIG. 13 shows an effect of SAC on an area of gastric lesions of rats induced by aspirin, wherein *** indicates a significant difference from a vehicle control group G1 by p<0.001, G1: vehicle control group (distilled water), G2: 100 mg/kg of SAC, G3: 200 mg/kg of SAC, G4: 400 mg/kg of SAC, and G5: positive control group (55.6 mg/kg of Stillen® as an active ingredient);

FIG. 14 shows gastric lesion inhibiting rates in rats in an aspirin-induced gastric lesion model;

FIG. 15 shows photographs of stomachs of rats in an aspirin-induced gastric lesion model;

FIG. 16 shows an effect of SAC on lengths of gastric lesions of rats induced by indomethacin, wherein * indicates significant difference from a vehicle control group G1 by p<0.05, G1: vehicle control group (distilled water), G2: 100 mg/kg of SAC, G3: 200 mg/kg of SAC, G4: 400 mg/kg of SAC, and G5: positive control group (55.6 mg/kg of Stillen® as an active ingredient);

FIG. 17 shows gastric lesion inhibiting rates in rats in an indomethacin-induced gastric lesion model; and

FIG. 18 shows photographs of stomachs of rats in an indomethacin-induced gastric lesion model.

### DETAILED DESCRIPTION OF THE INVENTION

### Mode for the Invention

Hereinafter, the present invention will now be described more fully with reference to the accompanying drawings, in which exemplary embodiments of the invention is shown.

A composition including S-allyl-L-cysteine (SAC) has an excellent anti-Helicobacter pylori activity and gastric mucosa protective effect.

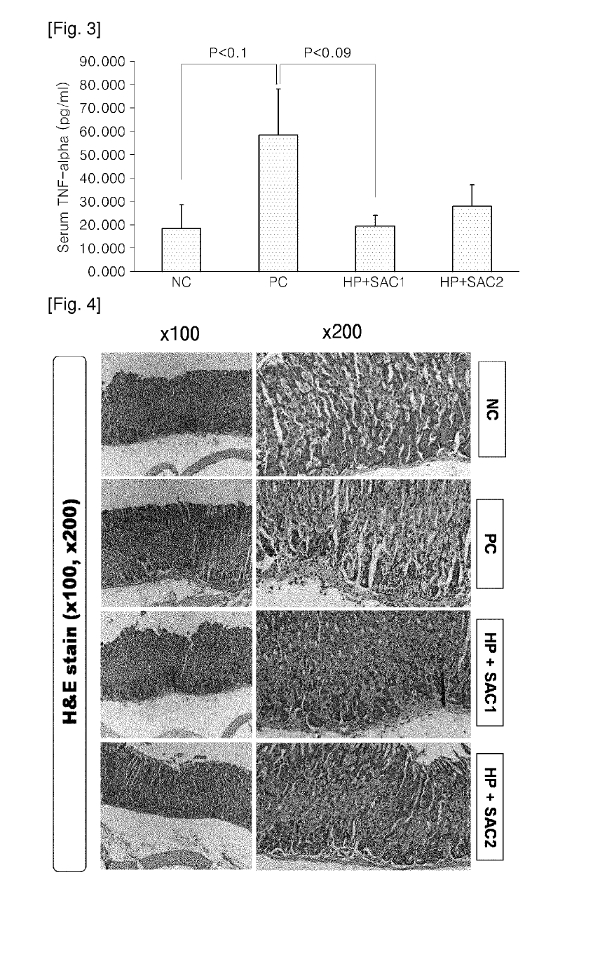

The present inventors have found that a positive control group of mice to which Helicobacter pylori is administered has a significantly greater antibody titer (anti-H. pylori IgG) compared to a negative control group of mice to which Helicobacter pylori is not administered (p<0.01), but an experimental group to which SAC and Helicobacter pylori are administered has a significantly less Helicobacter pylori IgG compared to the positive control group (FIGS. 2A and 2B). This result indicates that SAC has an effect of inhibiting mice from being infected with Helicobacter pylori. The present inventors have also found that the amount of TNF-α, which is an inflammation factor associated with T cells, increased in mice of the positive control group to which Helicobacter pylori was administered but was reduced if SAC was administered with Helicobacter pylori (FIG. 3). As a result, it can be seen that SAC inhibits inflammation caused by Helicobacter pylori infection in mice.

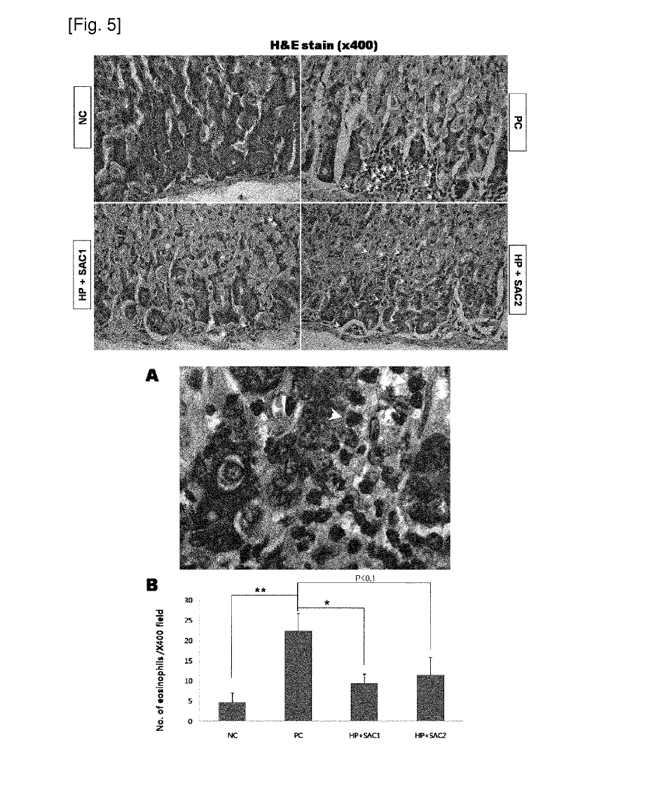

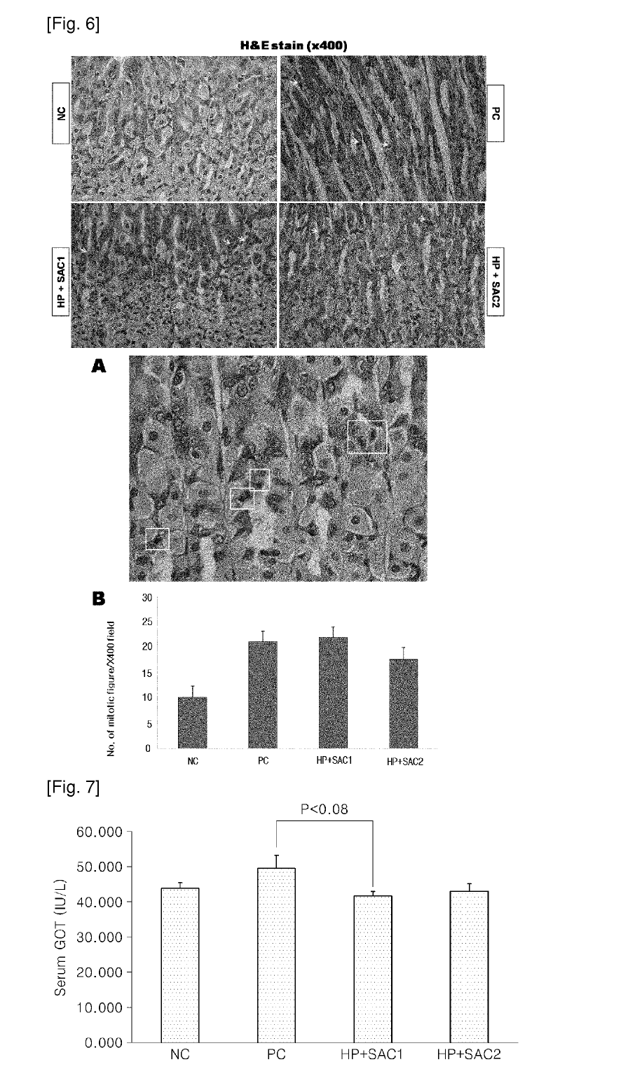

In order to observe a SAC effect on gastric lesions caused by Helicobacter pylori infection, sections of mouse stomach tissue were stained with hematoxylin and eosin (H&E staining). As a result, in groups to which Helicobacter pylori was administered, denaturation of gastric mucosa cells and eosinophil infiltration in lamina propria were observed (FIG. 4), and the number of eosinophils infiltrated into gastric mucosal epithelium in a positive control group that was infected with Helicobacter pylori was greater than that of a negative control group that was not infected (p<0.01) but the number was significantly reduced in the experimental group to which SAC was administered (p<0.05, FIG. 5). The number of mitotic figures that represent cell nuclei undergoing cell division was also increased in the Helicobacter pylori-infected groups compared to the negative control group but reduced in SAC-administered groups (FIG. 6). As a result, it can be seen that gastric lesions caused by Helicobacter pylori can be prevented or healed by SAC.

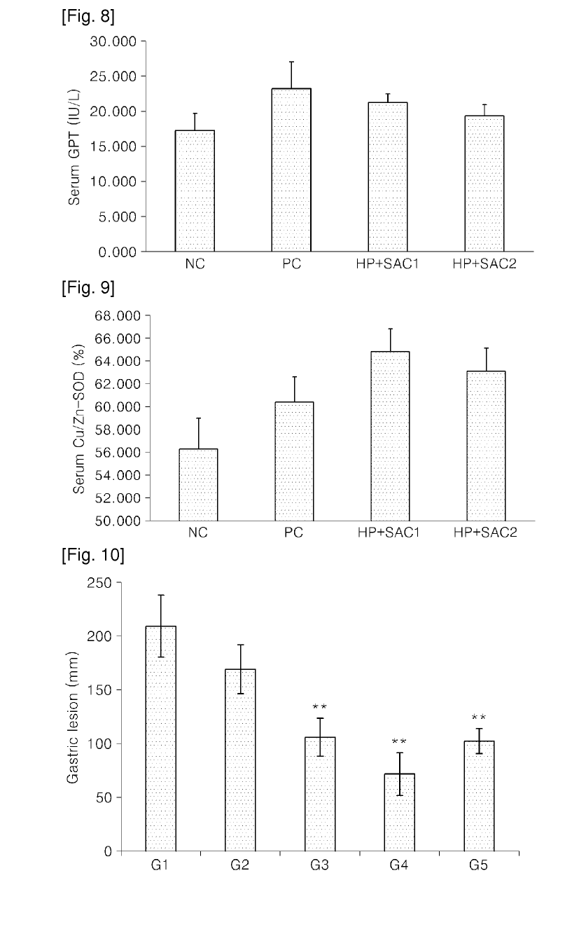

As a result of analyzing effects of SAC on serum biochemical levels of animals, glutamic oxaloacetic transaminase (GOT) and glutamate pyruvate transaminase (GPT) levels were the lowest in the negative control group that was not infected, and the GOT and GPT level of the SAC-administered group was less than the positive control group (FIGS. 7 and 8).

Copper and zinc containing-superoxide dismutase (Cu/Zn-SOD) level in serum was measured in order to observe effects of SAC on oxidative lesions. As a result, the Cu/Zn-SOD levels were increased in the Helicobacter pylori-infected groups compared to the negative control group that was not infected. However, the Cu/Zn-SOD level of the experimental group to which SAC were administered with was higher than that of the positive control group to which Helicobacter pylori was administered (FIG. 9). It was identified that SOD that was expressed by a defense mechanism against Helicobacter pylori infection was increased by SAC.

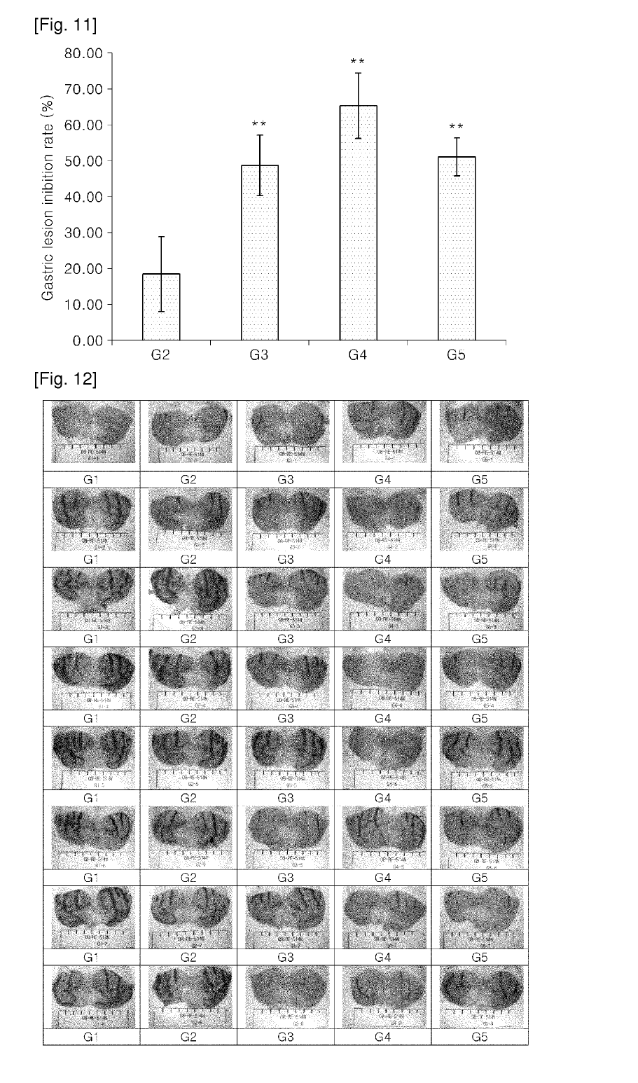

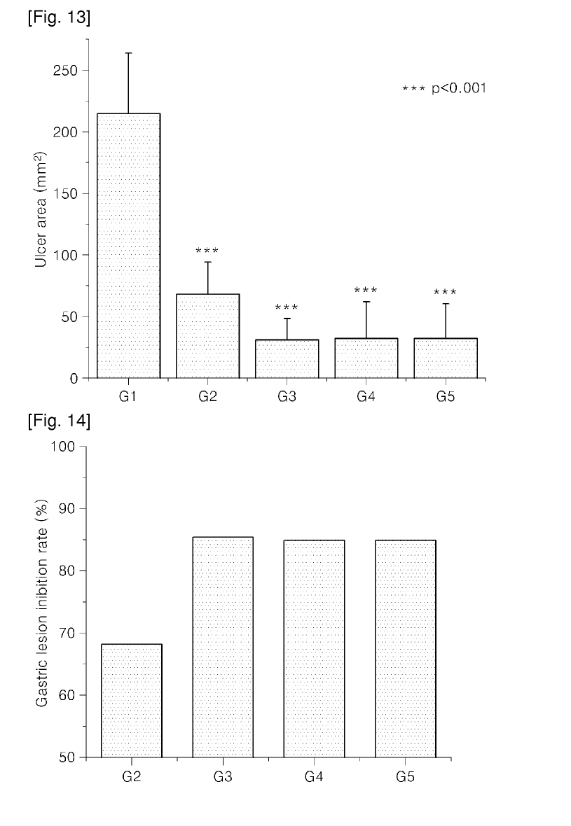

In addition, the present inventors have found that SAC has a significant effect on inhibiting gastric mucosal lesions that were induced in the rats by a drug administration. In a gastric lesion induced in a rat by hydrochloric acid-ethanol, SAC exhibited a significant reduction in the lesion when compared to a control group to which only a vehicle was administered (FIGS. 10 and 12) and a gastric lesion inhibiting rate (%) up to about 66% (FIG. 11). SAC also exhibited a significant reduction in a gastric lesion induced by aspirin (FIGS. 13-15) or indomethacin (FIGS. 16-18) when compared to the negative control group. SAC has a gastric lesion inhibiting rate (%) up to about 85% in the gastric lesion induced by aspirin and a gastric lesion inhibiting rate (%) up to about 94% in the gastric lesion induced by indomethacin, which are similar or better gastric mucosa protective effects compared to Stillen® used as a positive control group. The results indicate that SAC has diverse gastric mucosa protective effects on gastric lesion caused by various factors.

The present invention provides a pharmaceutical composition including SAC as an active ingredient with a pharmaceutically acceptable carrier, determined to have an anti-Helicobacter pylori activity based on the results of experiments.

The present invention provides a pharmaceutical composition including SAC as an active ingredient with a pharmaceutically acceptable carrier and having a gastric mucosa protective effect.

The present invention provides a food composition including SAC as an active ingredient and having an anti-Helicobacter pylori activity and a gastric mucosa protective effect.

The present invention provides a method of inhibiting Helicobacter pylori infection and preventing gastric lesions caused by Helicobacter pylori infection by using a composition including SAC as an active ingredient.

The present invention provides a pharmaceutical composition including SAC as an active ingredient and a pharmaceutically acceptable carrier for preventing or treating gastrointestinal disorders.

The present invention provides a food composition including SAC as an active ingredient for preventing or relieving gastrointestinal disorders.

The composition according to the present invention may further include a therapeutic agent for gastrointestinal disorders or an anti-Helicobacter pylori agent in addition to SAC.

The present invention provides a method of preventing or treating gastrointestinal disorders using a composition including SAC as an active ingredient and a pharmaceutically acceptable carrier.

Helicobacter pylori infection or gastrointestinal disorders are also common in animals, and thus the present invention also provides a composition for animals.

The gastrointestinal disorders to which the composition according to the present embodiment may be applied include chronic gastritis, acute gastritis, gastric ulcer, gastric cancer, bleeding in the gastrointestinal tract, gastroesophageal reflux disease (GERD), duodentis, and duodenum ulcers, but are not limited thereto.

The anti-Helicobacter pylori activity may include preventing or treating hepatic encephalopathy, arteriosclerosis, hepatobiliary system-associated diseases, urticaria, migraine, short stature, infertility, food allergy, chronic gastritis, acute gastritis, gastric ulcer, gastric cancer, bleeding in gastrointestinal tract, gastroesophageal reflux disease (GERD), duodentis, or duodenum ulcer, but is not limited thereto.

In the composition according to the present embodiment, a pharmaceutically or sitologically acceptable salt of SAC may be used as an active ingredient. The salt may be an acid addition salt or a quaternary ammonium salt. Examples of the acid addition salt include inorganic acid addition salts such as chloride, hydrobromide, hydroiodide, sulfate, and phosphate and organic acid addition salts such as oxalate, maleate, fumarate, lactate, malate, succinate, tartrate, benzoate, and methanesolfonate. Examples of the quaternary ammonium salt are a short-chain alkyl halogenide such as methyl iodide, methyl bromide, ethyl iodide, and ethyl bromide; a short-chain alkyl sulfonate such as methyl methanesulfonate and ethyl methanesulfonate; and a short-chain alkyl arylsulfonate such as methyl-p-toluenesulfonate.

The SAC or the pharmaceutically or sitologically acceptable salt thereof may exist in a solvate or hydrate form, and thus a solvate or hydrate of the SAC or the pharmaceutically or sitologically acceptable salt thereof may be used as an active ingredient for the therapeutic composition according to the present embodiment.

The SAC used herein may be prepared from a plant belonging to the Allium genus such as garlic, elephant garlic, onion, orscallion using a method disclosed in European Patent Publication No. EP 0429080A1, synthesis, fermentation, or any other known method.

The SAC, the pharmaceutically or sitologically acceptable salt of SAC, or a solvate or hydrate thereof, as an active ingredient, may be directly administered to patients. However, a composition including one or more of the active ingredients may be administered or a combined formulation prepared by mixing the active ingredients with an anti-Helicobacter pylori agent or a drug for treating gastrointestinal disorders may also be administered to patients.

The present invention provides a pharmaceutical composition formulated into a formulation for oral administration, a formulation for mucosal administration, an injection formulation, a formulation for inhalation, and a formulation for external application, but the formulation is not limited thereto. The formulation for oral administration may include hard and soft capsules, tablets, suspensions, powders, suspended-release formulations, enteric formulations, granules, oleosacchara, fine granules, pills, extracts, liquids, aromatic waters, emulsions, syrups, elixirs, fluid extracts, infusodecoctions, tinctures, medicated spirits, and infused oils, but is not limited thereto. The formulation for mucosal administration may be troches, buccal tablets, sublingual tablets, suppositories, and intranasal sprays, but is not limited thereto. The injection formulation may be subcutaneous injections, intramuscular injections, intravenous injections, and implant tablets, but is not limited thereto. The formulation for external application may be nasal drops, ophthalmic solutions, otic solutions, ophthalmic ointments, pastes, cataplasma, liniments, lotions, sprays, dusting powder, and liquids for external use, but is not limited thereto.

The formulation according to the present embodiment may further include one or more inert carriers, in addition to one or more active ingredients, for example, excipients such as starch, lactose, carboxymethyl cellulose, and kaolin, binders such as water, gelatin, alcohol, glucose, gum Arabic, and gum Tragacanth, disintegrants such as starch, dextrin, and sodium alginate, lubricants such as talc, stearic acid, magnesium stearate, and liquid paraffin, and other additives such as solubilizing agents.

A daily dose of SAC may vary according to various factors such as severity of disease, onset of the disease and a patient's age, condition, and complications. In general, a daily dose of SAC for an adult may be in the range of 1 mg to 10 g, preferably 100 mg to 4 g, and more preferably 200 to 2,000 mg. However, the daily dose may further increase for patients having severe symptoms or complications to improve therapeutic efficiency. The formulations may be administered in a single dose or divided into doses administered 2 or 3 times per day. For example, one or two unit dose formulations, each containing 200 to 500 mg of SAC may be orally administered once or twice per day, but the administration may be adjusted if required.

If the composition according to the present embodiment is a food composition, the amount of the active ingredients may be adjusted, if desired, for example, as disease-preventing or treating food or health supplements. In general, the amount of SAC in food or beverage may be in the range of 0.0001 to 90% by weight, preferably 0.1 to 50% by weight of the total food or beverage. Even though the amount of SAC in health supplements may be within the range described above for long-term use, it may be increased since the active ingredient is safe. Food including the food composition according to the present embodiment may be meat, sausage, bread, chocolate, candy, snack, pizza, instant noodle, gum, dairy products, soup, beverages, tea, drinks, alcohol, and vitamins, but is not limited thereto. Hereinafter, one or more embodiments will be described in detail with reference to the following examples. However, these examples are not intended to limit the purpose and scope of the invention.

### EXPERIMENTAL EXAMPLES

### I. Effect of SAC in Animal Infected with Helicobacter pylori

Test Material

S-allyl-L-Cysteine (SAC) was purchased from TCI Chemical Co. (Tokyo, Japan). Helicobacter pylori was American Type Culture Collection (ATCC) 43504 (cagA+, vacA s1-ml type) and cultured in a Mueller Hinton-Agar broth at 37° C. for 48 hours, under 5% CO₂ microaerophilic conditions with a concentration of 1×10⁹ CFU/ml.

Test Animal

8-week old male specific pathogen free (SPF) C57BL/6 mice were used. Weights of the mice were measured in the Animal Lab, Department of Pathology, the College of Veterinary Medicine, Kyungbook National University. Then, the mice were classified into 4 groups and bred such that average weights of each group are similar to each other. The mice were acclimated and bred in the Animal Lab, Department of Pathology, the College of Veterinary Medicine, Kyungbook National University at a temperature of 22±3° C., at a relative humidity of 50±10%, with light for 12 hours (light turned on at 08:00 and light turned off at 20:00) by an automatic temperature and humidity control system. Other environments for breeding which might influence the test for the entire test period were not considered for use. The mice were given free access to a solid laboratory diet (PMI Nutrition International, 505 North 4th Street Richmond, Ind. 47374, USA) and filtered tap water with water bottles.

Test Group and Administration

8-week old male C57BL/6 mice were classified into 4 groups, i.e., a positive control group (to which Helicobacter pylori was administered; PC), a negative control group (to which saline was administered; NC), experimental group 1 (to which Helicobacter pylori and 200 mg/kg of SAC were administered; SAC1), and experimental group 2 (to which Helicobacter pylori and 400 mg/kg of SAC were administered; SAC2). Each group had 10 mice. White powdered SAC was diluted in tap water to concentrations of 20 mg/mL and 40 mg/mL and 10 μl/g (body weight) of the diluted SAC was orally administered to the mice of the SAC1 and SAC2 groups three times a week for 10 weeks. The mice were given free access to tap water for the entire test period. The Helicobacter pylori was collected with saline to a concentration of 1×10⁹ CFU/mL, and 0.2 mL of the diluted Helicobacter pylori was orally administered to each mouse for 8 weeks from 2 weeks after SAC administration. The mice were fasted for 8 hours before the infection, and 0.15 mL of 0.2 M sodium bicarbonate (NaHCO₃) was administered to each mouse 10 minutes before the administration of Helicobacter pylori in order to neutralize acidified stomach due to the fasting. The same amount of saline instead of the infectious substances was administered into the NC group. The mice of all groups were fed with a normal diet. After a 10-week test period, autopsies were performed on all mice, and samples of blood and internal organs were collected for tissue pathological analyses. The test was performed according to the following process.

Statistical Method

Statistical significance of the obtained data was tested using an independent sample t-test. Statistical analysis was performed using SPSS 14.OK, and a p-value of less than 0.05 was considered significant.

### Experimental Example 1

### Effect of SAC on Weight

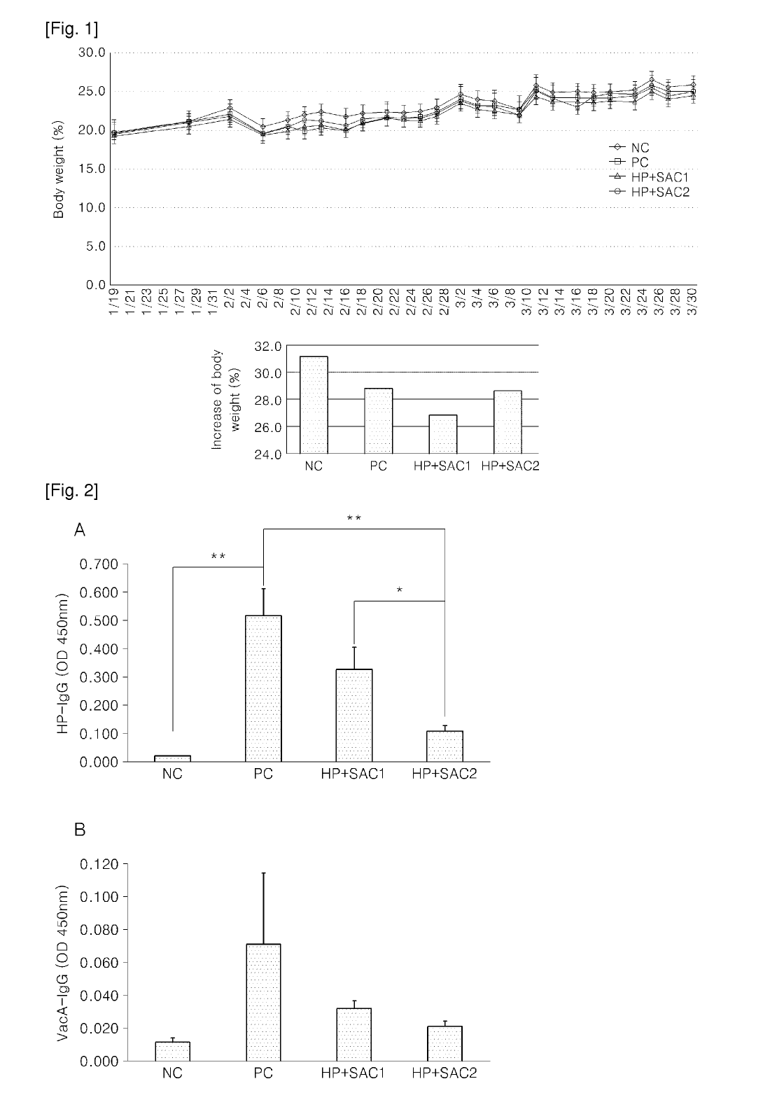

Weights of male C57BL/6 mice infected with Helicobacter pylori were measured three times a week for the entire 10-week test period to observe weight changes of the mice. The weights of the mice gradually increased in all groups, except that the weights were slightly and temporarily reduced at the time of Helicobacter pylori infection and acquisition of serum to identify infection (FIG. 1). A weight increase rate of the NC group was 31.3%, and those of the PC group, SAC1 group, and SAC2 group were respectively 28.9%, 26.9%, and 28.7%. The weight increase rate of the PC group was less than that of the NC group by 2.4%, the weight increase rate of the SAC1 group was less than the PC group by 2%, and the weight increase rate of the SAC2 group was less than that of the PC group by 0.2%. Even though the weight increase rate of the SAC2 group was greater than that of the SAC1 group by 1.8%, this result is deemed to be insignificant, and thus the Helicobacter pylori infection and test materials did not significantly influence the weight changes.

### Experimental Example 2

### Effect of SAC on Serum Anti-Helicobacter pylori (anti-H. pylori IgG) Antibody-Formationcapability

Method

Enzyme-linked immunosorbent assay (ELISA) was used in order to identify the effect of SAC on serum anti-Helicobacter pylori antibody-formation capability. Orally infectious H. pylori ATCC 43504 and a recombinant toxin VacA specifically producing Helicobacter pylori were used as antigens in mice. They were added to a 96-well microplate for analysis to concentrations of 1 μg/well and 10 ng/well, respectively, and the micro plate was maintained at 4° C. for coating. A supernatant was removed and a blocking buffer (1% skim milk) was added to the micro plate to inhibit unnecessary reactions, and the micro plate was maintained at 37° C. for 1 hour. 10 μl of serum of mice of all groups were added thereto and maintained at 37° C. for 2 hours. The microplate was washed with a Tris buffer solution including Tween20, and anti-mouse IgG, as a secondary antibody, bound to horse radish peroxidase (HRP) was added to the micro plate. Then, the microplate was maintained at 37° C. for 1 hour. Then, after being washed in the same manner, 100 μl of a mixture including a chromophore reagent of 3,3′,5,5′-tetramethylbenzidine (TMB) and the same amount of H₂O₂ was added thereto and the micro plate was maintained in dark conditions. As a result, coloring was identified within 30 minutes. 100 μl of 0.2 M sulfuric acid was added thereto to terminate the reaction and absorbance was measured at 450 nm.

Result

As a result of measuring an anti-H. pylori IgG antibody levelin serum, it was identified that the antibody against Helicobacter pylori (anti-H. pylori IgG) was produced in the PC group, the SAC1 group, and the SAC2 group to which Helicobacter pylori were administered and was not produced in the NC group to which Helicobacter pylori was not administered (FIGS. 2A and 2B). Meanwhile, an antibody titer of anti-H. pylori IgG in the SAC1 and SAC2 groups was less than that in the PC group, and the antibody titer in the SAC2 group was significantly less than that in the SAC1 group. Accordingly, the production of the antibody against Helicobacter pylori was inhibited in a SAC concentration-dependent manner (FIG. 2A). In addition, the production of the antibody against Helicobacter pylori-producing toxin VacA (anti-s1m1 VacA IgG) was inhibited in the same manner as in the anti-H. pylori IgG (FIG. 2B). As a result, it was identified that Helicobacter pylori infection was inhibited by SAC in a concentration-dependent manner.

### Experimental Example 3

### Effect of SAC on Serum TNF-α

Method

In order to observe the effect of SAC on an inflammation factor of TNF-α in serum, a commercially available mouse TNF-α antibody analysis kit Microplate (R&D systems Inc., USA) was used and concentrations of TNF-α in serum of mice were measured according to the manuals of the kit.

Result

The TNF-α value was increased in Helicobacter pylori-administered groups when compared to the NC group (p<0.1). The TNF-α of the SAC1 and SAC2 groups was less than that of the PC group (p<0.09) (FIG. 3). As a result, it was identified that SAC inhibits inflammation caused by Helicobacter pylori infection in mice.

### Experimental Example 4

### Effect of SAC on Histological Change of Stomach Caused by Helicobacter pylori

Method

Among lesions caused by Helicobacter pylori infection and occurring for the 8 week-test period, tissue pathological changes of a stomach were observed in a male C57BL/6 mouse Helicobacter pylori infection model. A stomach sample was fixed with 10% formalin and a paraffin block was formed. The paraffin block was cut into sections having a thickness of 4 μm, and the section were stained with hematoxylin-eosin (H&E staining) and observed using an optical microscope. All pathological features such as the severity of cell lesions, the number of infiltrated eosinophils, and the number of mitotic figures in the entire stomach were double-checked. In addition, the number of eosinophils was checked from a region where the esophagus and stomach join and counted from three regions of the stomach, i.e., cardia, gastric pit, and gastric crypt of lamina propria. The number of mitotic figures was counted from two regions selected from the entire antrum from cardia to pylorus. Thus two samples were obtained from each animal and evaluated with a 400× magnification, and an average value of each group was calculated.

Result

As a result of H&E staining, the infiltration of eosinophils and the number of the mitotic figures were observed in all groups. A number of eosinophils were found in lamina propria of the stomach and eosinophils infiltrated into gastric mucosal epithelium was observed (FIG. 4). The number of eosinophils was significantly increased in the Helicobacter pylori-infected groups compared to the NC group (p<0.01, FIG. 5B). On the other hand, the number of eosinophils in the SAC1 and SAC2 groups was less than that of the PC group, and particularly, the number of eosinophilsin SAC1 group was significantly less than that of the PC group (p<0.05, FIG. 5B). The number of the mitotic figures was also increased in the Helicobacter pylori-infected groups compared to the NC group. Even though there was no significant difference among the PC group, the SAC1 group, and the SAC2 group, the number of the mitotic figures was slightly reduced in the SAC2 group. The increase of eosinophil infiltration in the stomach, caused by Helicobacter pylori infection, has been reported in diverse previous research. The reduction in eosinophil infiltration by SAC indicates that SAC has protective effects against gastric lesions caused by Helicobacter pylori infection.

### Experimental Example 5

### Effect of SAC on Serologic Parameters in Helicobacter pylori-Infected Mouse Model

As a result of analyzing serum glutamic oxaloacetic transaminase (GOT) that is an index related to general lesions, the PC group exhibited the highest GOT level. Even though the SAC1 and SAC2 groups were not significantly different from the PC group, the GOT levels of the SAC1 and SAC2 groups were less than that of the PC group (p<0.08, FIG. 7).

As a result of analyzing serum glutamate pyruvate transaminase (GPT) that is an index related to liver lesion, the PC group exhibited the highest GPT level. Even though the SAC1 and SAC2 groups were not significantly difference from the PC group, the GOT level of the SAC1 and SAC2 groups was less than that of the PC group (FIG. 8).

### Experimental Example 6

### Measurement of Serum Cu/Zn-SOD Level

Method

Effect of SAC on copper and zinc containing-superoxide dismutase (Cu/Zn-SOD) that is an antioxidant enzyme in serum was observed in a male C57BL/6 mouse Helicobacter pylori infection model for the entire 10-week test period. In this regard, a superoxide dismutase activity assay kit (BioVision, Mountain View, Calif., USA) was used. 20 μl of serum and 200 μl of a WST working solution as a substrate were mixed. 20 μl of an enzyme working solution was added thereto, and the mixture was maintained at 37° C. for 20 minutes. Absorbance was measured at 450 nm. A control reaction was conducted in the same manner as described above, except that 20 μl of distilled water was added thereto instead of serum. SOD activity (inhibition rate %) was calculated.

Result

The Cu/Zn-SOD levels of the SAC1 and SAC2 groups were greater than that of the PC group by about 4% and about 3%, respectively. It was observed that the Cu/Zn-SOD levels of the SAC2 group were slightly less than the SAC1 group. Accordingly, it was identified that SAC promotes expression of SOD that is produced by a defense mechanism against Helicobacter pylori infection.

### II. Gastric Mucosal Protective Effect of SAC in Animals Having Gastric Lesions Caused by Drugs

The gastric mucosal protective effect of SAC was evaluated in animals having gastric lesions induced by hydrochloric acid-ethanol, aspirin, or indomethacin.

Test Material

SAC was purchased from TCI Chemical Co. (Tokyo, Japan). Sterile water for injection (Model No. 73H5F21, Dae Han Pharmaceutical Co. Ltd.) was used as a vehicle, and Stillen® was used as a positive control material. 0.5% CMC-Na and sterile water for injection were used as excipients for Stillen®. Hydrochloric acid was purchased from Samjung Chemical, Co., ethanol was purchased from Baker, Co., aspirin was purchased from Sigma, Co., and indomethacin was purchased from Sigma, Co.

Test Animals

7 to 8-week old male specific pathogen free (SPF) HsdKoat:Sprague-Dawley®™ SD®™ rats (weight of 7-week old males was in the range of 208.44 to 227.39 g, and weight of 8-week old males was in the range of 223.85 to 245.03 g, purchased from Koatech, Co. Ltd., Gyunggido, Korea) were quarantined and acclimated in the Animal Lab for 7 days. The rats were bred at a temperature of 23 3° C., at a relative humidity of 55 15%, with light for 12 hours (light turned on at 08:00 and light turned off at 20:00) while air was ventilated 10 to 20 times/hr in the Animal Experiment Lab of Gyeonggi Bio-Center. Other environments for breeding which might influence the test for the entire test period were not considered for use. The rats were given free access to a solid laboratory diet (Harlan Co. Ltd., USA. Teklad certified global 18% protein rodent diet, 2918C) that was supplied by Folas International. According to the analysis of certificate of diet composition, there were no ingredients or contaminants that could have had an adverse effect on the test. The rats were given free access to tap water that was sterilized using a UV sterilizer and a micro filter with water bottles.

Test Group and Administration

The rats were classified into a vehicle control group G1 to which only a vehicle was administered, a experimental group G2 to which 100 mg/kg of SAC was administered, a experimental group G3 to which 200 mg/kg of SAC was administered, a experimental group G4 to which 400 mg/kg of SAC was administered, and a positive control group G5 to which 100 mg/kg of Stillen® (55.6 mg/kg as an active ingredient) as a positive control material were administered. Each group included 8 rats in a hydrochloric acid-ethanol-induced animal model (Experiment Example 7) and included 6 rats in an aspirin-induced animal model (Experimental Example 8) and in an indomethacin-induced animal model (Experimental Example 9).

TABLE 1

| | | Total | | Adminis- | |
|---|---|---|---|---|---|
| | | Number of | Number of | tered Vol- | Dose |
| Group | Gender | animals | animals | ume(ml/kg) | (mg/kg) |
| G1 | Male | 8(6) | 1~8(1~6) | 10 | 0 |
| G2 | Male | 8(6) | 9~16(7~12) | 10 | 100 |
| G3 | Male | 8(6) | 17~24(13~18) | 10 | 200 |
| G4 | Male | 8(6) | 25~32(19~24) | 10 | 400 |
| G5 | Male | 8(6) | 33~40(25~30) | 10 | 55.6^{a)} |

G1: Vehicle control group
G2 to G4: Experimental groups to which SAC is administered
G5: Positive control group to which a positive control material is administered
^{a)}Dose of active ingredient

The test substance was directly administered to the stomach using an injection tube equipped with a sonde for oral administration in a single dose once per day.

No animals died and no other changes were observed in any of the groups, and significant weight change with respect to the administration of the SAC was not observed at the administration and for the entire test period.

Statistical Method

Comparisons of the vehicle control group with the experimental groups and the positive control material-administered experimental group were verified using one-way analysis of variance (One-way ANOVA). In this regard, significance and homoscedasticity were accepted, and thus a post-doc test was conducted using a Duncantest. Significance was accepted when p<0.05, and SPSS 10.1 was used.

### Experimental Example 7

### Effect of SAC on Lengths of Gastric Lesions and Gastric Lesion Inhibiting Rate in Hydrochloric Acid-ethanol-induced Gastric Lesion Animal Model

Method

The test substances were administered. After one hour, 1.5 ml of 150 mM HCl in 60% ethanol was orally administered to each rat. The rats to which ethanol and SAC were administered were fasted without water in stainless steel breeding cages for 1 hour. After one hour from the hydrochloric acid-ethanol administration, the rats were sacrificed under anesthesia using ether and their stomachs including parts of the duodenum and esophagus were isolated. The insides of the stomachs were immediately washed with 13 ml of a 2% neutral buffered formalin, and the duodenum and esophagus parts were fixed with forceps. Then, 13 ml of the 2% neutral buffered formalin was added thereto and maintained for 5 minutes for fixation. The greater curvature of each stomach was incised, fixed to a dissection table, and unfolded to measure the lengths of gastric lesions using vernier calipers. Photographs of the unfolded stomachs were taken (FIG. 12) and the stomachs were fixed with a 10% neutral buffered formalin.

Result

According to this gastric lesion model, ethanol directly stimulates gastric mucosa, induces edema in a muscle layer under a mucous membrane to cause a transient ischemic condition, and thus cell necrosis is induced due to oxidative damage, and hydrochloric acid directly stimulates the gastric mucosa and accelerates gastric motility to cause acute gastritis. By gross finding, the lesion was observed over the entire gastric mucosa and hemorrhage was observed in a long line. After one hour from the hydrochloric acid-ethanol administration, the rats of the vehicle control group had lesions over the entire gastric mucosa which was identified by atopsy (209.60±28.39 mm). The experimental group to which 200 mg/kg of SAC was administered (106.65±16.70 mm, p<0.01), the experimental group to which 400 mg/kg of SAC was administered (72.25±19.33 mm, p<0.01), and the positive control group to which Stillen® was administered (102.51±11.35, p<0.01) exhibited significantly less lesions than the vehicle control group (refers to FIGS. 10 and 12).

Gastric lesion inhibiting rate (%)=(average length of the vehicle control group−length of gastric lesion of each animal)/average length of the vehicle control group×100.

The experimental group to which 200 mg/kg of SAC was administered (41.12±7.97%, p<0.01), the experimental group to which 400 mg/kg of SAC was administered (65.53±9.22%, p<0.01), and the positive control group to which Stillen® was administered (51.09±5.41%, p<0.01) exhibited a significantly greater gastric lesion inhibiting rate (%) than the vehicle control group (refers to FIG. 11).

### Experimental Example 8

### Effect of SAC on Area of Gastric Lesion and Gastric Lesion Inhibiting Rate in Aspirin-induced Gastric Lesion Animal Model

Method

The rats were fasted for more than 24 hours in a normal environment, the test substance was administered, and 200 mg/kg of aspirin in 0.15 mol/L HCl was orally administered after 30 minutes. After three hours from the aspirin administration, the rats were sacrificed under ether anesthesia and their stomachs including parts of the duodenum and esophagus were isolated. The stomachs were was fixed for 10 minutes by injecting 12 ml of 2% formalin. The greater curvature of each stomach was incised and unfolded, photographs of the gastric grandular region were taken, and then the areas of the lesions were measured using an image analyzer.

Result

TABLE 2

| | Animal number (n = 6) | | | | | | UlcerArea | | |
|---|---|---|---|---|---|---|---|---|---|
| Group | 1 | 2 | 3 | 4 | 5 | 6 | (mm²) | SD | IR (%) |
| G1 | 165.76 | 1555.77 | 216.14 | 231.88 | 285.72 | 236.67 | 215.3 | 48.35 | |
| G2 | 91.68 | 44.12 | 40.95 | 63.52 | 63.52 | 64.67 | 68.6 | 25.94 | −68.2 |
| G3 | 46.29 | 19.37 | 5.41 | 27.07 | 27.07 | 43.86 | 31.4 | 16.99 | −85.4 |
| G4 | 63.26 | 9.34 | 70.28 | 40.03 | 40.03 | 11.58 | 32.5 | 29.78 | −84.9 |
| G5 | 16.86 | 29.58 | 7.82 | 71.91 | 71.91 | 61.96 | 32.5 | 28.09 | −84.9 |

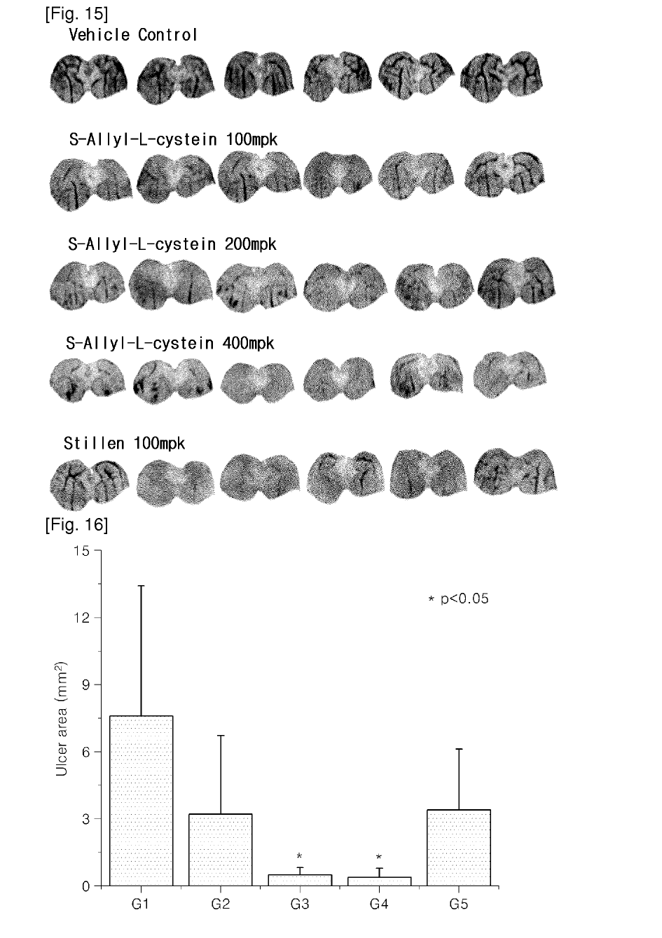

According to this gastric lesion model, aspirin that is a nonsteroidal anti-inflammatory drug inhibits the synthesis of prostaglandin that protects stomach walls, thereby causing gastric ulcers. The lesions were observed over the entire gastric mucosa and bleeding was observed in a net form. After aspirin administration, the rats of the vehicle control group had bleedings and lesions over the entire gastric mucosa which was identified by autopsy (215.3±48.35 mm²). The experimental group to which 100 mg/kg of SAC was administered (68.6±25.94 mm²), the experimental group to which 200 mg/kg of SAC was administered (31.4±16.99 mm²), the experimental group to which 400 mg/kg of SAC was administered (32.5±29.78 mm²), and the positive control group (32.5±28.09 mm²) exhibited significantly less lesions than the vehicle control group (refers to FIGS. 13 and 15).

Gastric lesion inhibiting rate (%)=(average area of the vehicle control group−area of gastric lesion of each animal)/average area of the vehicle control group×100.

The experimental group to which 100 mg/kg of SAC was administered (68.2%), the experimental group to which 200 mg/kg of SAC was administered (85.4%), the experimental group to which 400 mg/kg of SAC was administered (84.9%), and the positive control group (84.9%) exhibited a significantly greater gastric lesion inhibiting rate (%) than the vehicle control group (refer to FIG. 14).

### Experimental Example 9

### Effect of SAC on Area of Gastric Lesion and Gastric Lesion Inhibiting Rate in Indomethacin-induced Gastric Lesion Animal Model

Method

The rats were fasted for more than 24 hours in a normal environment, SAC was administered, and 25 mg/kg of indomethacin in distilled water was orally administered after 30 minutes. After six hours from the indomethacin administration, the rats were sacrificed under ether anesthesia and their stomachs including parts of the duodenum and esophagus were isolated. The stomachs were fixed for 10 minutes by injecting 12 ml of 2% formalin, the greater curvature of each stomach was incised and unfolded, photographs of the lesions were taken, and then the areas of the lesions were measured using an image analyzer.

Result

TABLE 3

| | Animal number (n = 6) | | | | | | UlcerArea | | |
|---|---|---|---|---|---|---|---|---|---|
| Group | 1 | 2 | 3 | 4 | 5 | 6 | (mm²) | SD | IR (%) |
| G1 | 6.4 | 18.0 | 6.7 | 4.2 | 0.9 | 9.6 | 7.6 | 5.85 | |
| G2 | 1.3 | 1.6 | 1.5 | 9.4 | — | 2.3 | 3.2 | 3.47 | −57.9 |
| G3 | 0.0 | 0.3 | 0.9 | 0.6 | 0.7 | 0.4 | 0.5 | 0.30 | −93.8 |
| G4 | 0.8 | 0.4 | 0.1 | 0.2 | 0.1 | 1.0 | 0.4 | 0.36 | −94.4 |
| G5 | 3.7 | 0.6 | 2.7 | 0.7 | 7.7 | 5.2 | 3.4 | 2.75 | −55.2 |

According to this gastric lesion model, indomethacin that is a nonsteroidal anti-inflammatory drug inhibits the synthesis of prostaglandin that protects stomach walls, thereby causing gastric lesions. Local lesions were observed in the gastric mucosa and bleeding was observed. After indomethacin administration, the rats of the vehicle control group had lesions having an area of 7.6±5.85 mm² which was identified by autopsy. In the experimental group to which 100 mg/kg of SAC was administered, the area of the lesions was 3.2±3.47mm² which was less than that of the vehicle control group by a half or more. The experimental group to which 200 mg/kg of SAC was administered (0.5±0.30 mm²), the experimental group to which 400 mg/kg of SAC was administered (0.4±0.36 mm²), and the positive control group (3.4±2.75 mm²) exhibited significantly less lesions than the vehicle control group. In particular, the experimental groups to which 200 mg/kg and 400 mg/kg of SAC were administered exhibited light gastric lesions and no bleedings (FIGS. 16 and 18).

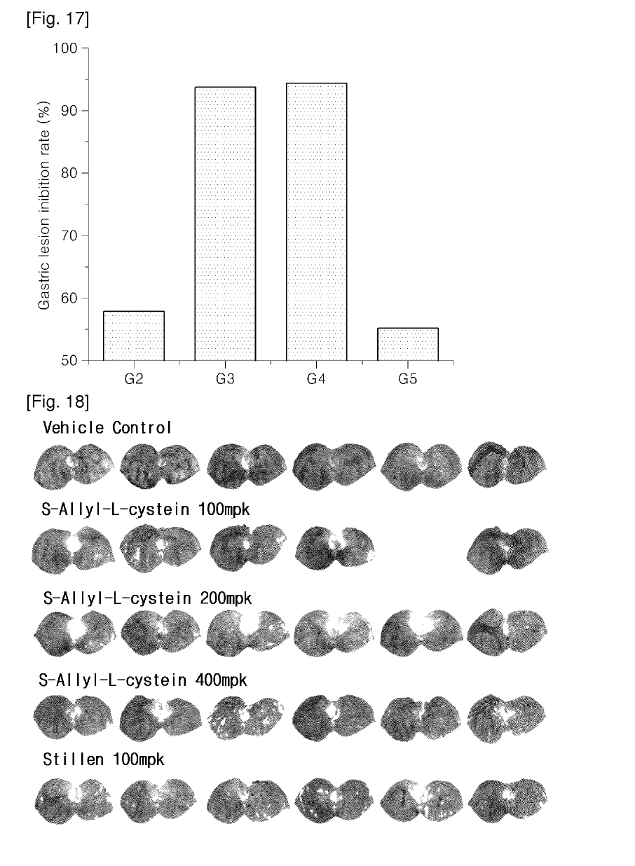

The gastric lesion inhibiting rate (%) was calculated in the same manner as in Experimental Example 8. The experimental group to which 100 mg/kg of SAC was administered (57.9%), the experimental group to which 200 mg/kg of SAC was administered (93.8%), the experimental group to which 400 mg/kg of SAC was administered (94.4%), and the positive control group (55.2%) exhibited a significantly greater gastric lesion inhibiting rate (%) than the vehicle control group. Particularly, it was identified that the gastric lesion may be completely inhibited if 200 mg/kg or more of SAC is administered (refers to FIG. 17).

### PREPARATION EXAMPLES

Various formulations including SAC as an active ingredient were prepared as follows.

### Preparation Example 1

### Manufacture of Tablet

SAC 200 mg

Lactose 50 mg

Starch 10 mg

Magnesium stearate appropriate quantity

The ingredients above were mixed and tableted using a known method to prepare a tablet.

### Preparation Example 2

### Manufacture of Powder

SAC 250 mg

Lactose 30 mg

Starch 20 mg

Magnesium stearate appropriate quantity

The ingredients above were mixed and filled in a chartula coated with polyethylene, and the chartula was sealed to prepare powder.

### Preparation Example 3

### Manufacture of Capsule

SAC 500 mg

Lactose 30 mg

Starch 28 mg

Magnesium stearate appropriate quantity

The ingredients above were mixed and filled in a gelatin hard capsule using a known method to prepare a capsule.

### Preparation Example 4

### Manufacture of Suspension

SAC 50 mg

Isomerized sugar 10 g

Sugar 30 mg

Sodium carboxymethyl cellulose 100 mg

Lemon flavor appropriate quantity

Total volume including purified water 100 ml

The ingredients above were mixed to prepare a suspension using a known method and the suspension was filled in a 100 ml-brown bottle and sterilized.

### Preparation Example 5

### Manufacture of Soft Capsule (Amount in One Soft Capsule)

SAC 500 mg

Polyethylene glycol 400 400 mg

Concentrated glycerin 55 mg

Purified water 35 mg

Polyethylene glycol and concentrated glycerin were mixed, and purified water was added thereto. Flavone was added thereto while the mixture was maintained at about 60° C., and the mixture was stirred at about 1,500 rpm with a stirrer. The mixture was cooled to room temperature while slowly stirring and bubbles were removed using a vacuum pump to prepare contents for a soft capsule. The coating of the soft capsule was prepared using gelatin and a plasticizer which are known in the art. 132 mg of gelatin, 52 mg of concentrated glycerin, 6 mg of 70% disobitol solution, an appropriate amount of ethyl vanillin as a flavoring agent, and carnauba wax as a coating base were used to prepare one soft capsule using a known method.

### Preparation Example 6

### Manufacture of Injection

SAC 200 mg

Mannitol 180 mg

Sterilized distilled water for injection 2974 mg

Na₂HPO₄12H₂O 26 mg

An ample having the ingredients above was prepared using a known method.

### Preparation Example 7

### Manufacture of Beverage

SAC 0.01 g

Citric acid 8.5 g

White sugar 10 g

Glucose 2.5 g

DL-malic acid 0.3 g

Purified water appropriate quantity

The ingredients above and an appropriate amount of purified water were mixed to a total volume of 100 mL and stirred to prepare a beverage using a known method.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A pharmaceutical composition comprising S-allyl-L-cysteine, a pharmaceutically acceptable salt of S-allyl-L-cysteine, or a solvate or hydrate thereof as an active ingredient and having an anti-Helicobacter pylori activity.

2. The pharmaceutical composition of claim 1, wherein the anti-Helicobacter pylori activity comprises activities that prevent or treat hepatic encephalopathy, arteriosclerosis, hepatobiliary system-associated diseases, urticaria, migraine, short stature, infertility, food allergy, chronic gastritis, acute gastritis, gastric ulcer, gastric cancer, bleeding in gastrointestinal tract, gastroesophageal reflux disease, duodentis, or duodenum ulcer.

3. A pharmaceutical composition comprising S-allyl-L-cysteine, a pharmaceutically acceptable salt of S-allyl-L-cysteine, or a solvate or hydrate thereof as an active ingredient for preventing or treating gastrointestinal disorders.

4. The pharmaceutical composition of claim 3, wherein the gastrointestinal disorders are selected from the group consisting of chronic gastritis, acute gastritis, gastric ulcer, gastric cancer, bleeding in gastrointestinal tract, gastroesophageal reflux disease, duodentis, and duodenum ulcer.

5. A pharmaceutical composition comprising S-allyl-L-cysteine, a pharmaceutically acceptable salt of S-allyl-L-cysteine, or a solvate or hydrate thereof as an active ingredient and having a gastric mucosa protective effect.

6. The pharmaceutical composition of claim 1, wherein the S-allyl-L-cysteine is isolated from a plant belonging to the Allium genus and purified, synthesized, or prepared by fermentation.

7. A pharmaceutical formulation comprising a composition according to claim 1, the pharmaceutical formulation selected from the group consisting of a formulation for oral administration, a formulation for mucosal administration, an injection formulation, a formulation for inhalation, and a formulation for external application.

8. The pharmaceutical formulation of claim 7, wherein the formulation for oral administration is selected from the group consisting of hard and soft capsules, tablets, suspensions, powder, suspended-release formulations, enteric formulations, granules, oleosacchara, fine granules, pills, extracts, liquids, aromatic waters, emulsions, syrups, elixirs, fluid extracts, infusodecoctions, tinctures, medicated spirits, and infused oils.

9. A food composition comprising S-allyl-L-cysteine, a salt of S-allyl-L-cysteine, or a solvate or hydrate thereof as an active ingredient and having an anti-Helicobacter pylori activity or a gastric mucosa protective effect.

10. A food composition comprising S-allyl-L-cysteine, a salt of S-allyl-L-cysteine, or a solvate or hydrate thereof as an active ingredient for preventing, relieving, or treating gastrointestinal disorders.

11. The food composition of claim 10, wherein the gastrointestinal disorders are selected from the group consisting of chronic gastritis, acute gastritis, gastric ulcer, gastric cancer, bleeding in gastrointestinal tract, gastroesophageal reflux disease, duodentis, and duodenum ulcer.
